(19) United States
(12) Patent Application Publication
(10) Pub. No.: US 2003/0199749 A1
(43) Pub. Date: Oct. 23, 2003

(54) Uterine Magnetomyography

(75) Inventors: Curtis L. Lowery JR., Little Rock, AR (US); Hari Eswaran, Little Rock, AR (US); Pamela M. Murphy, North Little Rock, AR (US); James D. Wilson, Benton, AR (US)

Correspondence Address: Christine J. Daugherty, WRIGHT, LINDSEY & JENNINGS LLP, Suite 102, 320 North Rollston, Fayetteville, AR, 72701, US

(73) Assignee: Board of Trustees of the University of Arkansas

(21) Appl. No.: 10/411,027

(22) Filed: Apr. 9, 2003

Related U.S. Application Data
(60) Provisional application No. 60/373,466, filed on Apr. 17, 2002.

(51) Int. Cl. G01R 33/02; A61B 5/05

(52) U.S. Cl. 600/409; 324/248

(57) ABSTRACT

The present invention is directed to satisfying the need to measure and monitor uterine activity non-invasively and accurately. Using superconducting quantum interference device sensors, we have established the feasibility of recording uterine contractile activity with high enough spatial-temporal resolution to determine the regions of localized activation and propagation over the uterus. With the large surface area and the shape of the array, spatial-temporal recordings of uterine activity were obtained using 151 sensors yielding a better insight into the mechanism of uterine contraction. By obtaining a contour plot of the magnetic field distribution, we were able to localize the areas of activation over the uterus during a contraction.

### CROSS-REFERENCE TO RELATED APPLICATIONS

The present application claims the benefit of U.S. Provisional Application No. 60/373,466, filed Apr. 17, 2002, which is incorporated herein by reference.

### STATEMENT REGARDING FEDERALLY SPONSORED RESEARCH OR DEVELOPMENT

Not applicable.

### BACKGROUND OF THE INVENTION

1. Field of the Invention

The present invention relates to non-invasively recording the uterine magnetomyographic activity using a complete spatial-temporal map of uterine activity, to predict the onset of term labor and the presence of preterm labor.

2. Brief Description of the Related Art

At this time, our knowledge of the physiological mechanism of the onset and propagation of uterine contractions of labor remains incomplete. Unwanted hospital stays and treatment can be avoided if physicians are able to more accurately predict the onset of labor and differentiate true labor from false labor both for term and preterm patients. The lack of a truly effective method for diagnosis of labor points to the need for a new, innovative investigation into the physiology of uterine activity.

The uterine electromyography (EMG)—a measure of electrical activity of the uterus—has been studied using both internal electrodes and abdominal surface electrodes (Larks et al., 1957, J Appl Physiol, 10: 479-83; Hon and Davis, 1958, Obstet Gynecol, 12: 47-53; Kuriyama and Csapo, 1961, Endocrinology, 68: 1010-25; Csapo and Takeda, 1963, Nature, 200: 680-2; Sureau et al., 1965, Bull. Fed Soc Gynec Obstet., 17(1): 79-140; Wolfs and Rottinghuis, 1970, Arch Gynak, 208: 373-85; Wolfs and Van Leeuwen, 1979, Acta Obstset Gynecol Scan Suppl, 90:1-61; Zahn, 1984, J Perinatol Med, 12:107-13; Marque et al., 1989, Applied Biosensors. Stoneham: Butterworth; p. 187-226; Devedeu et al., 1993, Am J of Obstet Gynecol, 169: 1636-53; Buhimschi et al., 1997, Obstet Gynecol., 90:102-111; Garfield et al., 1998, J of Perinat Med., 26(6): 413-36; Garfield et al., 1998, Human Reproduction Update, 4(5): 673-95; Germain et al., 1982, Am J Obstet Gynecol., 142: 513-19; Buhimschi et al., 1998, Am J Obstet Gynecol., 178: 811-22). Past research has shown that the uterine myometrial activity is low throughout pregnancy but significantly increases during term or preterm labor (Wolfs and Van Leeuwen, 1979, Acta Obstset Gynecol Scan Suppl, 90:1-61; Zahn, 1984, J Perinatol Med, 12:107-13; Marque et al., 1989, Applied Biosensors. Stoneham: Butterworth; p. 187-226; Devedeu et al., 1993, Am J of Obstet Gynecol, 169: 1636-53). As pointed out by Garfield, the earlier studies could not conclusively determine if the electrical activity recorded at the abdominal surface was a true representation of myometrial electrical activity (Garfield et al., 1998, J of Perinat Med., 26(6): 413-36; Garfield et al., 1998, Human Reproduction Update, 4(5): 673-95). Their group recently performed simultaneous recording of the EMG activity directly from the uterus and from the abdominal surface of rats (Buhimschi et al., 1998, Am J Obstet Gynecol., 178: 811-22). They proved that the EMG activity recorded from the rat's abdominal surface mirrors the activity generated in the uterus.

Using multiple electrodes, Steer et al. and Sureau et al. have tried to map the topography of the electrical activity of the uterus (Steer et al., 1950, Am J of Obstet Gynecol 59:25-40; Sureau et al., 1965, Bull. Fed Soc Gynec Obstet., 17(1): 79-140). Steer et al placed two pairs of electrodes overlying each fallopian tube junction and a third pair high in the mid-line of the fundus (Steer et al., 1950, Am J of Obstet Gynecol 59:25-40). They reported that a weak activity picked by one of the two pairs of electrodes showed a small time lag in early labor and the lag diminished as the labor progressed. During labor they observed that the activity from all the three pairs of electrodes were almost simultaneous.

Further, electromyography studies performed by Garfield et al. show that there is infrequent and unsynchronized low uterine electrical activity throughout most of pregnancy (Buhimschi et al., 1997, Obstet Gynecol., 90:102-111; Garfield et al., 1998, J of Perinat Med., 26(6): 413-36; Garfield et al., 1998, Human Reproduction Update, 4(5): 673-95; Germain et al., 1982, Am J Obstet Gynecol, 142: 513-19). However, at term, changes in the uterine physiology result in better propagation and synchronization of electrical burst activity throughout the uterus causing rhythmic contractions leading to the delivery of the fetus. All these studies show that the progress of labor is related to the propagation of electrical activity throughout the uterus. Thus the efficiency of contractions leading to labor depends on the synchronous burst activity over a large area of the uterus. Therefore, it is important to determine the velocity and the extent of propagation throughout the multi-cellular uterine muscle bundle. Since the propagation of these uterine contractions can occur in both longitudinal and transverse direction, we must determine the propagation characteristics over the entire maternal abdomen while performing surface recordings. We believe that information gained from the analysis of the spatial-temporal activation of the uterus may be predictive of onset of term labor and the presence of preterm labor. Thus, a complete spatial-temporal mapping of uterine activity throughout pregnancy is a key parameter that will improve the understanding of the uterine contraction mechanism. In order to improve the spatial-temporal resolution, we studied the feasibility of performing non-invasive magnetic field recordings—magnetomyography (MMG)—of the uterus with the use of the 151 channel SARA (SQUID Array for Reproductive Assessment) system installed at the University of Arkansas for Medical Sciences (UAMS) hospital. SQUID is an acronym for Superconducting Quantum Interference Device.

All electrophysiological phenomena are characterized by the flow of ion currents within the body. These currents can be detected by measuring potentials inside or on the surface of the body. The physics of electromagnetism predicts that the flow of current will also result in a magnetic field. Consequently, common clinical electrophysiological measurements such as the electrocardiogram (ECG) and electroencephalogram (EEG) have magnetic homologues, the magnetocardiogram (MCG) and the magnetoencephalogram (MEG), respectively (Williamson et al., 1983, Biomagnetism: an interdisciplinary approach. New York-London: Plenum Press). It is well known that uterine EMG signals suffer some degree of attenuation during their propagation to the surface of the maternal abdomen. This attenuation is caused by differences in conductivity of the tissue layers. By contrast, magnetic field recordings are much less dependent on tissue conductivity and are detectable outside the boundary of the skin without making electrical contact with the body. Unlike electrical recordings, the magnetic recordings are independent of any kind of reference, thus ensuring that each sensor mainly records localized activity.

The above references describe utilizing uterine electromyography (EMG) to measure the electrical activity of the uterus. However, EMG signals suffer from attenuation caused by differences in conductivity of the tissue layers. Magnetic field recordings are much less dependent on tissue conductivity and are detectable outside the boundary of the skin without making electrical contact with the body. Therefore utilizing magnetic recordings of the uterus is a better tool to predictive of onset of term labor and the presence of preterm labor. The limitations of the prior art are overcome by the present invention as described below. References mentioned in this background section are not admitted to be prior art with respect to the present invention.

### BRIEF SUMMARY OF THE INVENTION

The present invention is directed to satisfying the need to measure and monitor uterine activity non-invasively and accurately. With the large surface area and the shape of the SARA array, we have demonstrated the capability of non-invasively recording the uterine magnetomyographic activity along with the requisite spatial-temporal resolution needed to study its propagation over the pregnant uterus. Unlike cardiac cells, there is no evidence of the existence of a fixed anatomic pacemaker area on the uterine muscle. It is believed that the action potential burst can originate from any uterine cell and the pacemaker site can shift from one contraction to another. Despite this shifting of the pacemaker site, it is possible to localize the pacemaker by mapping the magnetic field distribution during each contraction with sensors spread over the entire maternal abdomen. Furthermore, to study the propagation of the activity from the source, we can use the cross-correlation technique on the combined measurements from horizontal, vertical and diagonally oriented sensors to build a map of signal propagation. Once the propagation time is known the propagation velocity can be determined since the 3-D positional coordinate for each sensor is known.

An embodiment of the invention comprises non-invasively recording the uterine magnetomyographic activity via magnetic fields. The detailed spatial-temporal resolution of the SARA instrument will help to determine the regions of localized activation, propagation velocity, and direction and the spread of activity as a function of distance. This information may be predictive of onset of term labor and the presence of preterm labor. Utilizing the SARA system, signature characteristic that help differentiate between false and true labor can be identified. The present invention is not limited to utilizing a SARA system to obtain magnetic recordings. As the results of the analysis obtained by magnetic recordings can be used to improve the technical and data analysis aspects of the transabdominal EMG uterine monitoring.

### BRIEF DESCRIPTION OF THE DRAWINGS

These and other features, objects and advantages of the present invention will become better understood from a consideration of the following detailed description and accompanying drawings.

FIG. 1A is a view of the 151-channel SARA system with sensor array built to match the shape of a gravid abdomen.

FIG. 1B shows a pregnant mother positioned on a SARA system.

FIG. 2 shows uterine MMG signal recordings from 151 channels with strong uterine activity seen in the lower left side of the abdomen.

FIG. 3A shows an expanded view of the signals obtained from four sensors labeled MRF1, MRF2, MRG1 and MRG2 in the region of activity. The channel labeled STIM shows the time points of the beginning and the end of the contraction as perceived by the subject.

FIG. 3B shows location of the four sensors on the maternal abdomen.

FIG. 3C shows the frequency spectrum corresponding to the burst activity obtained from sensors.

FIG. 4A shows an expanded view of the MMG activity recorded from the three sensors under the region of maximum activity

FIG. 4B is a contour map of magnetic field distribution during a contraction at time point 176.56 sec.

FIG. 5A shows a sample MMG recording showing the propagation delay during a contraction obtained from six sensors.

FIG. 5B shows the position of the six sensors over the maternal abdomen.

FIG. 6 shows normalized cross-correlation sequence computed from data obtained from a pair of vertically (V) oriented sensors, MCE0 and MCI0, and a pair of horizontal (H) sensors, MLI1 and MRI1.

### DETAILED DESCRIPTION OF THE INVENTION

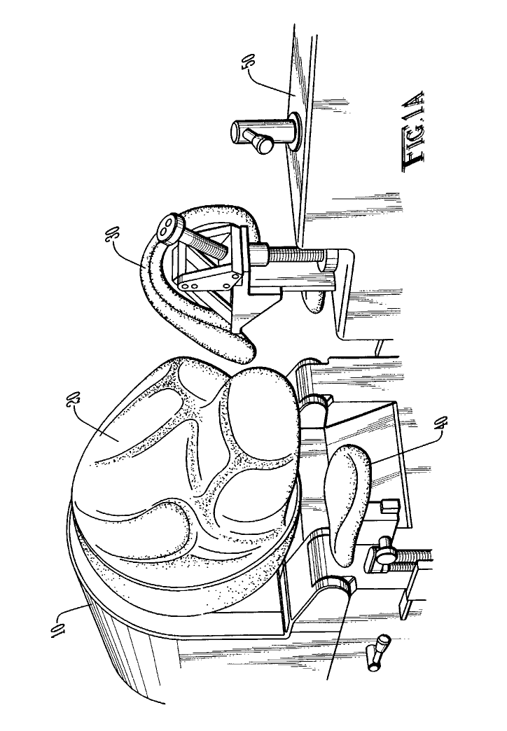

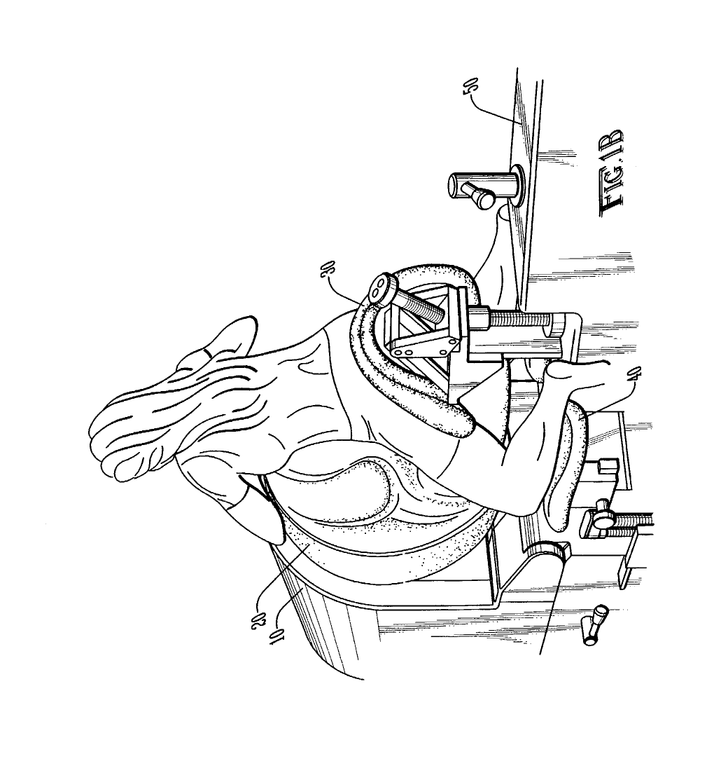

With reference to FIGS. 1-6, the preferred embodiment of the present invention may be described. The present invention is directed to satisfying the need to measure and monitor uterine activity non-invasively and accurately. Studies were performed to establish information that can be used to predict onset of term labor and the presence of preterm labor. With the large surface area and the shape of the SARA array, we have demonstrated the capability of non-invasively recording the uterine magnetomyographic activity along with the requisite spatial-temporal resolution needed to study its propagation over the pregnant uterus.

In this invention we measured magnetomyographic signals by utilizing a superconducting quantum interference device (“SQUID”) containing an array of SQUID sensors. One existing machine that posses an array of SQUID sensors is the SQUID Array for Reproductive Assessment (“SARA”) (CTF Systems Inc, Vancouver, Canada). The SARA system 10 consists of 151 primary magnetic sensors 20 spaced approximately 3 cm apart over an area of 850 cm². The sensors 20 are arranged in a concave array covering the maternal abdomen from the pubic symphysis to the uterine fundus, and laterally over a similar span. This array surface is curved to match the shape of the gravid abdomen. A pregnant patient 60 sits on the adjustable seat 30, which is located on top of the dewar 50. The pregnant patient 60 then places her legs in the adjustable leg rests 40 and leans forward against the smooth surface of the array allowing the SQUID sensors 20 to receive signals from the entire maternal abdomen. SARA system 10 which is installed in a magnetically shielded (Vakuumschmeize, Germany) room next to the labor and delivery unit in UAMS, has been operational since May 2000. The magnetic shielding reduces the influence of strong external magnetic fields that interfere with the biomagnetic fields generated by human organs. The purpose of the room is similar to that of a soundproof room or an electrically shielded room used for electrophysiological studies.

The subjects were ten pregnant mothers with gestational ages ranging from 29 to 40 weeks. This study was approved by Institutional Review Board of the hospital. After obtaining a written consent, the mothers were asked to sit comfortably and lean forward on to the sensor array. The mothers were asked to raise their finger for the duration of each perceived contraction. Based on this information, the operator synchronized the beginning and end of the contraction by marking these time points in the record. The recording sessions ranged from 12 to 28 minutes with a sampling rate of 250 Hz. The data was then down-sampled to 25 Hz and post-processed with a bandpass filter (0.05-1 Hz) for further analysis.

In order to localize the areas of activation over the uterus during a contraction, a contour map of the magnetic field distribution was plotted. The pattern of the field distribution helps in determining the area of activity over the uterus thus allowing for precise localization of the source of the MMG activity. In addition, as an initial step, we analyzed the recordings from the sensor array for delays in propagation time. To quantify the time delay between pairs of channels the normalized cross-correlation was computed. The cross-correlation functions measure the degree of similarity between two signals for arbitrary time delays. Completely synchronized channels will show a clear peak at a time delay of zero and every peak besides a zero time delay indicates a time delayed synchronized activation of the two channels. The sign of the time delay shows which channel is activated first.

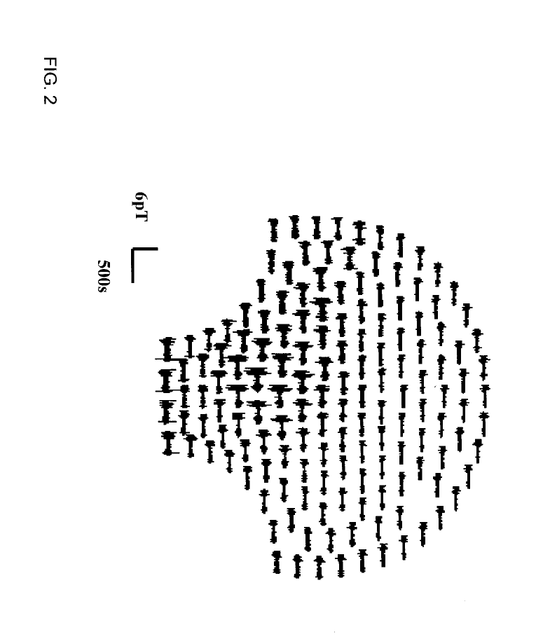

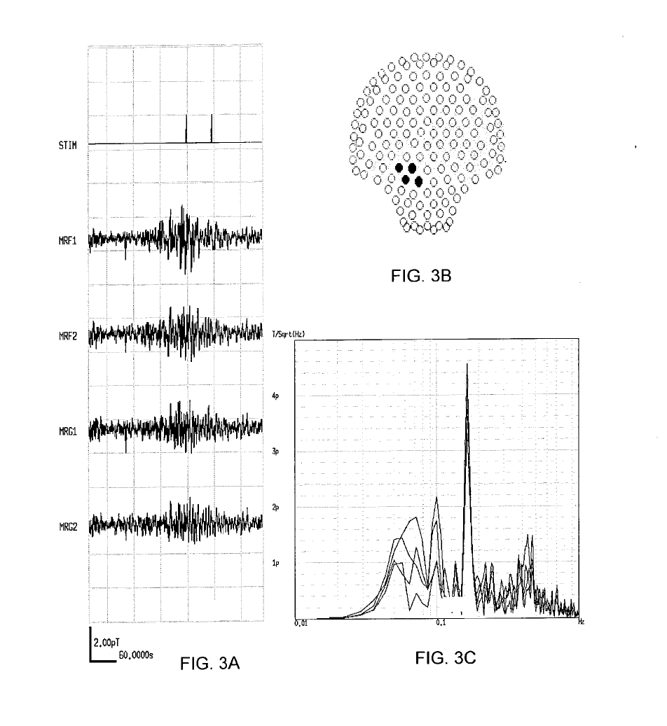

Table I shows the gestational ages of the ten subjects along with the number of uterine burst activity per minute observed by MMG recordings and by maternal perception. In all subjects except two, the number of bursts per minute obtained by these two methods is the same. Representative magnetomyographic activity obtained from 151 sensors covering the entire maternal abdomen is shown in FIG. 2. These recordings were acquired from a pregnant subject at 35 weeks of gestation. In this subject we can see strong uterine burst activity in the lower left region of the abdomen. FIG. 3A shows an expanded view of the signals obtained from four sensors in this along with the time point markers indicating the maternal preception of the contraction. It can be observed that the MMG burst activity starts a little earlier than when the mother feels the contraction. FIG. 3B shows the location of the four sensors on the maternal abdomen and FIG. 3C shows the frequency spectrum corresponding to the burst activity obtained from these sensors. In this subject, the frequency spectrum showed a dominant peak at 0.16 Hz with an amplitude of 4.5 pico Tesla (pT).

TABLE I
Comparison of the number of uterine contractions detected per minute
from 10 subjects by MMG and maternal perception.

Number of uterine contrac-
tions per minute obtained
from:

| Subject | Gestation | MMG | Maternal |
|---|---|---|---|
| number | age (weeks) | recordings | perception |
| 1 | 35 | 0.21 | 0.21 |
| 2 | 37 | 0.37 | 0.37 |
| 3 | 37 | 0.30 | 0.30 |
| 4 | 38 | 0.12 | 0.12 |
| 5 | 39 | 0.33 | 0.33 |
| 6 | 38 | 0.50 | 0.50 |
| 7 | 29 | 0.25 | 0.17 |
| 8 | 40 | 0.08 | 0.08 |
| 9 | 38 | 0.42 | 0.33 |
| 10 | 40 | 0.33 | 0.33 |

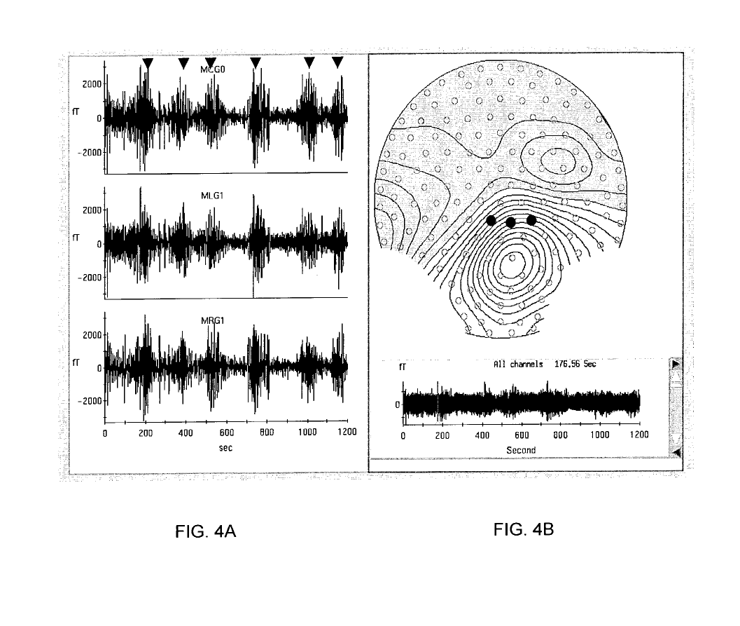

The contour map in FIG. 4B shows the magnetic field distribution at time point 176.56 sec and, plotted below it, is an entire 20-minute recording session of MMG activity from all the sensors. This recording was acquired from a subject at 37 weeks gestation. By visual inspection of the contour map, we can precisely localize the source of the activity over the uterus. FIG. 4A shows the expanded view of MMG data from three sensors in this region of maximum activity.

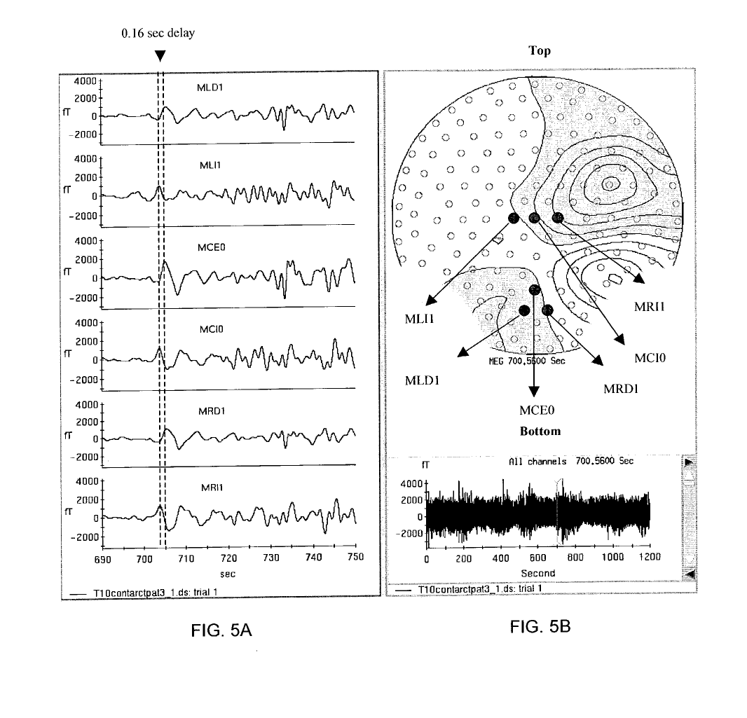

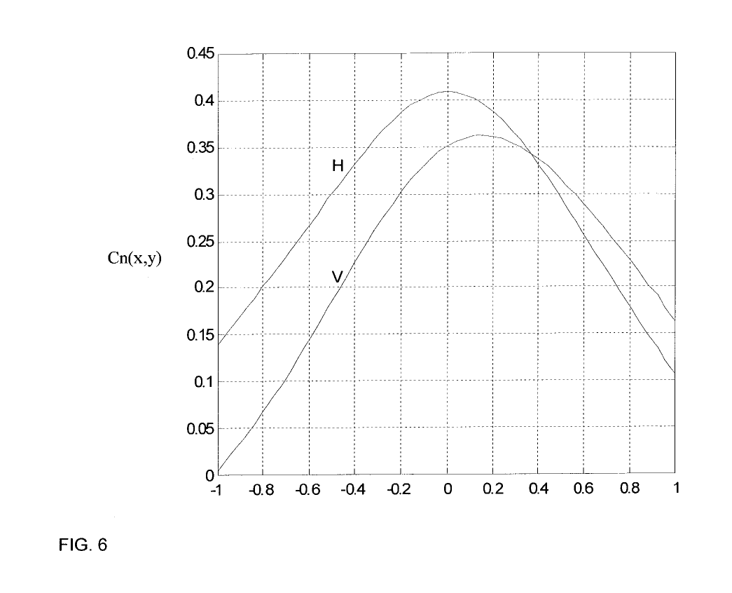

In order to study the feasibility of obtaining the propagation delay in the above data set, we picked six channels that are positioned as shown in FIG. 5B. A sample uterine contraction segment recorded by these channels is shown in FIG. 5A. From the figure it is evident that there is no delay in the horizontal direction along the channels MLI1, MCI0 and MRI1. Also, along the horizontal direction there is no delay across the channels MLD1, MCE0 and MRD1 whereas a delay of 0.16 sec. is observed in the vertical direction going from sensor MLI1 to MLD1, MCE0 to MCI0, and MRI1 to MRD1. The cross-correlation plot shown in FIG. 6 was obtained by using four centrally located sensors—one sensor pair aligned vertically—MCE0 and MCI0 (V) and the other pair aligned horizontally—MLI1 and MRI1 (H). The normalized cross-correlation was computed for each sensor pair. For the horizontal pair of sensors, the normalized cross-correlation function has a maximum at time zero, implying that the signals arrived at each sensor at the same time. In contrast, the normalized cross-correlation for the vertical pair clearly shows a time lag estimated to be about 0.16 sec. Therefore in this example, the contractile signals appear to be progressing in a vertical direction.

In summary, the detailed spatial-temporal resolution of the SARA instrument will help to determine the regions of localized activation, propagation velocity, and direction and the spread of activity as a function of distance. Overall, the advantages of this invention can be used as a predictive tool for the onset of term labor and the presence of preterm labor.

The present invention has been described with reference to certain preferred and alternative embodiments that are intended to be exemplary only and not limiting to the full scope of the present invention as set forth in the appended claims.

What is claimed is:

1. A non-invasive method for characterizing uterine magnetomyographic activity comprising:
a) providing a system consisting of a plurality of superconducting quantum interference device sensors shaped in a configuration adapted to a gravid abdomen of a pregnant patient;
b) placing said gravid abdomen of said pregnant patient in proximity to said sensors;
c) collecting from said sensors magnetomyographic signals produced by electrophysiological activity of uterine muscle cells of said pregnant patient;
d) analyzing said magnetomyographic signals produced by said electrophysiological activity of uterine muscle cells;
e) characterizing uterine activity of said pregnant patient based on an analysis of said magnetomyographic signals.

2. The method of claim 1, wherein said sensors are horizontal, vertical, and diagonally oriented in a concave array to collect said magnetomyographic signals.

3. The method of claim 1, further comprising, the step of cross-correlation analysis to calculate propagation time and velocity of said magnetomyographic signals.

4. The method of claim 3, wherein said calculated propagation time and velocity of said magnetomyographic signals is used to produce a spatial temporal propagation map of changing magnetic field distribution.

5. The method of claim 1, further comprising, the step of diagnosing labor as a function of said analyzed magnetomyographic signals produced by the electrophysiological activity of uterine muscle cells of said pregnant patient.

6. The method of claim 5, wherein said diagnosis step comprises predicting term labor.

7. The method of claim 5, wherein said diagnosis step comprises predicting preterm labor.

8. The method of claim 5, wherein said diagnosis step comprises an obstetrical diagnosis.

9. The method of claim 5, wherein said diagnosis step comprises an obstetrical treatment plan.
